(19) United States
(12) Patent Application Publication
(10) Pub. No.: US 2005/0004470 A1
(43) Pub. Date: Jan. 6, 2005

(54) Apparatus for mounting at least one ultrasound head at a medical interventional facility

(76) Inventors: Estelle Camus, Erlangen (DE); Robert Jorg, Neunkirchen (DE); Mark Lothert, Exton, PA (US); Klaus Thormann, Forchheim (DE)

Correspondence Address: SCHIFF HARDIN, LLP;PATENT DEPARTMENT, 6600 SEARS TOWER, CHICAGO, IL, 60606-6473, US

(21) Appl. No.: 10/856,367

(22) Filed: May 28, 2004

(30) Foreign Application Priority Data
May 30, 2003 (DE) 203 08 606.6

(51) Int. Cl. A61B 8/00

(52) U.S. Cl. 600/459

(57) ABSTRACT

An apparatus for mounting at least one ultrasound head to a patient positioning table has a base element with at least one chamber for an electronic module, and with at least one attachment element for an ultrasound head. The attachment element has a chamber to accept an electronic module associated with the respective ultrasound head and a mounting or acceptor for the ultrasound head.

### BACKGROUND OF THE INVENTION

1. Field of the Invention

The present invention concerns an apparatus for mounting at least one ultrasound head on a patient positioning table to allow the ultrasound head near its location of use during short-term periods of non-use.

2. Description of the Prior Art

A diagnostic apparatus is known from German Patent 100 15 824 that has an x-ray device and an ultrasound system. Such a diagnostic acoustic pressure sensor with ultrasound technology is increasingly used since it does not make use of penetrating radiation and is simple to operate. For some years, the use of ultrasound systems during medical interventional procedures has proven to be an attractive tool. This enables the precise implementation of vessel and organ centeses with real-time imaging. Interventional procedures can be implemented faster and more precisely and complications can be prevented. Moreover, complex, minimally-invasive interventions can be implemented using ultrasound.

The ultrasound head must be available to the physician in the area of the patient positioning table and be covered so that they remain sterile. An advantageous position of the ultrasound heads would be directly on the table, where an accessory rail is already provided at which the operating elements for the instruments necessary for the intervention can be placed or mounted.

In European Application 752 237, an operating table is disclose at in which a number of such attachment rails are mounted circumferentially for acceptance of surgical accessories such as, for example, mountings for surgical instruments, infusion brackets, operating units for additional devices, etc. Such mountings, for example, are locked to the attachment rails by means of rail clamp devices, so they can be easily slid on the attachment rails by releasing the clamp.

German Patent 198 07 242, corresponding to U.S. Pat. No. 6,585,731, discloses a medical-technical system workspace for open or minimally-invasive surgery with a patient positioning table, with an ultrasound applicator arranged on an administering unit which, for example, is disposed such that it can shift on rails on the patient positioning plate. Under the table is the ultrasound apparatus that is connected with the ultrasound applicator via a connection line.

Normally, however, the ultrasound apparatus is too large, and the cable length between the electronics and the ultrasound heads too short, to realize a mechanical integration of the ultrasound apparatus in an angiography system in the interventional environment. With such an ultrasound apparatus it is not possible to house the ultrasound head at the table without situating the large electronics module in direct proximity to the table. This solution is unacceptable from the perspective of the manufacturer and from the point of view of the physician, and the ultrasound apparatus must then be made available as an independent apparatus in the interventional environment. In practice, the ultrasound apparatus is brought into the intervention room only when necessary. Bringing the ultrasound apparatus in and out is very complicated and disturbs the course of the interventional procedure. Moreover, the availability of the independent ultrasound apparatus is restricted since it may be needed simultaneously not only in one room, but in multiple rooms.

German OS 197 14 984 describes an integrated apparatus and operating unit for a surgical-treatment workspace with at least two apparatuses and/or apparatus components, a central control unit housed in the housing, a central operating unit with central supply connections for input/output of data/working media, and a central display unit. This integrated apparatus and operating unit is arranged on a stand, in particular a ceiling stand. A mount for a feeding or cleaning bottle is provided.

From German OS 36 17 235, a biopsy adapter is known that is connected to an ultrasound head with a swallowtail connection.

### SUMMARY OF THE INVENTION

An object of the present invention is to provide an apparatus of the initially-cited type that will enable a secure mounting of the ultrasound head to the patient positioning table as well as a direct accommodation of the ultrasound system in the proximity of the patient positioning table.

The object is inventively achieved by an apparatus with a base -element that has at least one chamber for a first electronic module, and at least one attachment element for an ultrasound head that has a chamber to accept an electronic module associated with the respective ultrasound head, and a mounting or acceptor for the ultrasound head.

The apparatus can be simply, detachably mounted to a patient positioning table in an embodiment wherein at least one of the elements is provided with at least one mounting clamp to mount to an accessory rail on the patient positioning table.

The base elements and mounting elements can be arbitrarily connected together as needed when they are laterally connected with one another such that they can be detached. The elements can be laterally connected with one another by swallowtail mechanisms (guides; tracks; rails).

As an alternative, the connection can be effected by an element having a P-shaped cutout, with the adjacent element having a knob, engaging in the cutout, with the head of the knob fitting through the wide opening of the cutout and the neck fitting in the narrow part of the cutout.

The first electronic module can be a voltage supply module.

The apparatus also can be attached to tilting tables when they have a level-regulating adapter. Such an adapter can be attached directly to the table.

A further alternative is for the mounting clamps to engage via a mounting-side adapter rail that is connected via a rotation element for level compensation with a table-side adapter piece that has a hook attachment for the accessory rail.

It has proven to be advantageous when one of the elements is a mounting for an ultrasound gel bottle.

The above object also is inventively achieved by an ultrasound apparatus with at least one ultrasound head to which a first electronic module is connected for the control of the ultrasound head and the acquisition of the image data, the head also being connected with a second electronic module for voltage supply, with a computer to reproduce the ultrasound images, and with a device to accept at least one ultrasound head.

### DESCRIPTION OF THE DRAWINGS

FIG. 1 is an overview of a first embodiment of an ultrasound head mounting.

FIG. 2 is an overview of a second embodiment of an ultrasound head mounting in accordance with the invention.

FIG. 3 shows a base element usable in both embodiments.

FIG. 4 is a separate view of a first attachment element, shown in FIG. 1.

FIG. 5 is a separate view of a second attachment element, shown in FIG. 2.

FIG. 6 is a perspective view of an ultrasound head mounting with the elements according to FIGS. 3 and 5.

FIG. 7 is a perspective view of an ultrasound head mounting in accordance with the invention attached to a patient positioning table.

FIG. 8 shows the electrical connection of an ultrasound head.

FIG. 9 is a side view of an ultrasound head mounting with a level-regulating rotation adapter in accordance with the invention.

FIG. 10 shows an element of the mounting according to FIG. 9.

FIG. 11 is a front view of the mounting according to FIG. 9.

FIG. 12 shows a level-regulating rotation adapter according to FIG. 9.

FIG. 13 shows a level-regulating ultrasound head mounting in accordance with the invention.

FIG. 14 shows a P-shaped cutout as a further embodiment for connection of the individual elements of an ultrasound head mounting in accordance with the invention.

FIG. 15 shows a knob as the counterpart for the connection according to FIG. 14.

### DESCRIPTION OF THE PREFERRED EMBODIMENTS

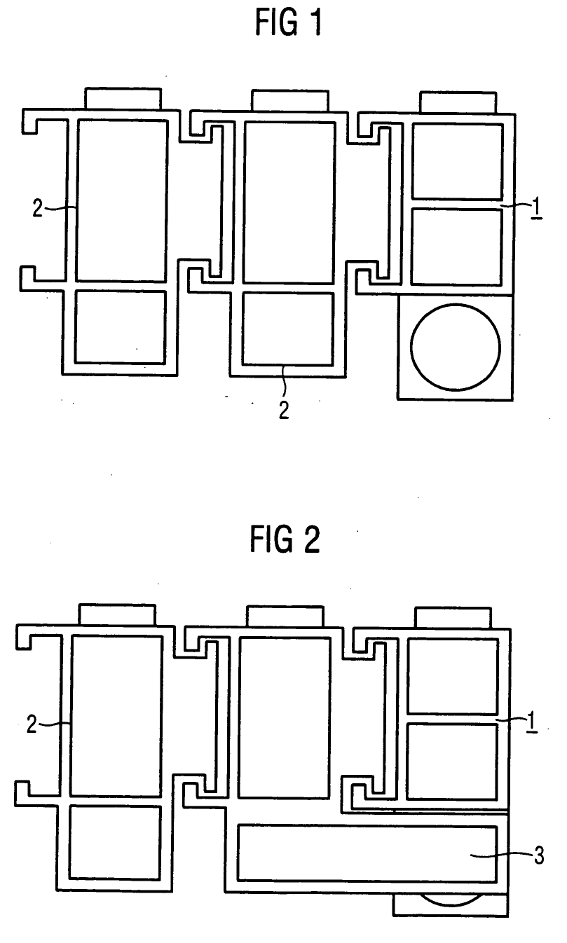

FIG. 1 shows an ultrasound head mounting having a base element and two uniformly fashioned attachment elements 2. The attachment elements 2 are provided for the mounting small ultrasound heads, as described below.

In FIG. 2, the middle attachment element 2 has been replaced by a further attachment element 3 that is fashioned to accept a large ultrasound head. Two different ultrasound heads thus can be used simultaneously with this arrangement.

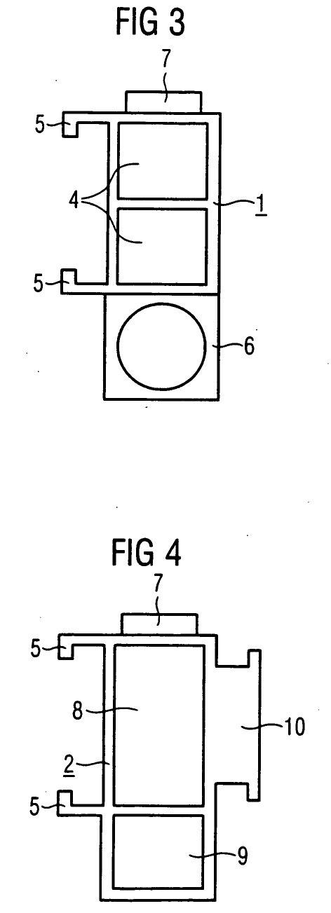

The individual elements 1 through 3 are shown in FIGS. 3 through 5. In FIG. 3, the base element 1 is shown that, for example, has two chambers 4. The base element itself exhibits a generally rectangular shape, as can be seen from FIG. 6. Both chambers 4 can be provided for the acceptance of a voltage supply module. On the left side, two L-shaped legs 5 engage a swallowtail mechanism with the corresponding counterparts of the other elements. A mounting 6 for an ultrasound gel bottle (shown below the chamber 2 in FIG. 3) is attached in front of the base element 1. Opposite this is a bracket 7 for attaching the base element 1 on an accessory rail (described below) of a patient positioning table.

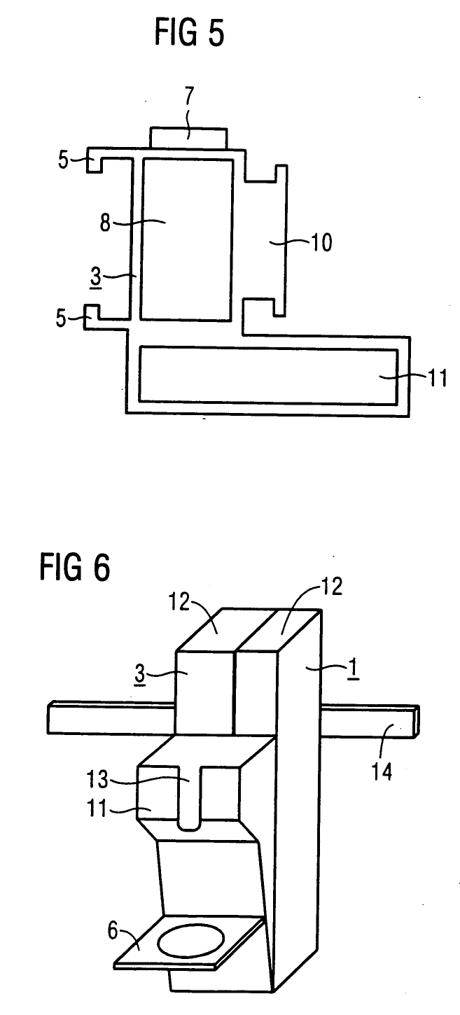

Shown in FIG. 4 is the first attachment element that has a chamber 8 for an electronics module of an ultrasound head. The acceptor 9 for a small ultrasound head is disposed in front of this, below the chamber 9 in FIG. 5. To the right of the attachment element, the counterpart 10 for the swallowtail connection is disposed that, as shown in FIGS. 1 and 2, is connected with the L-shaped legs 5 of the neighboring element. So that additional elements can be attached, the first attachment element 2 is also provided with L-shaped legs for the swallowtail connection.

The attachment element 3 for a large ultrasound head is shown in FIG. 5. It is essentially has the same elements as the attachment element 2, with only the exception being that the acceptor 11 is formed for a large ultrasound head and, as can be seen from FIG. 2, exhibits the width of the second attachment element 3 and the base element 1.

An inventive mounting composed of a base element 1 and the second attachment element 3 for a large ultrasound head is shown in perspective in FIG. 6. The chambers 4 and 8 of the elements 1 and 3 are covered by covers 12, such that the electronics modules located therein are not visible. As already stated, the acceptance 11 for the large ultrasound head is arranged over the entire width of both elements 1 and 3. The acceptor exhibits a slot 13 that allows that the cable of the large ultrasound head to hang out of the acceptor 11 given an inserted ultrasound head. Below the acceptance 11 is the mounting 6 for a bottle of ultrasound gel, which (depending on the dimensions of the elements) can exhibit the entire width of the elements, or only a part of the width. The mounting 6 can be attached to the base element 1, such that the mounting is always independent of the attached attachment elements 2 or 3. This can ensue by a bracket that is attached to this below the base element 1.

To mount the ultrasound head mounting, it is attached with the brackets 7 shown in FIGS. 1 through 5 to an accessory rail 14 of a patient positioning table (not shown).

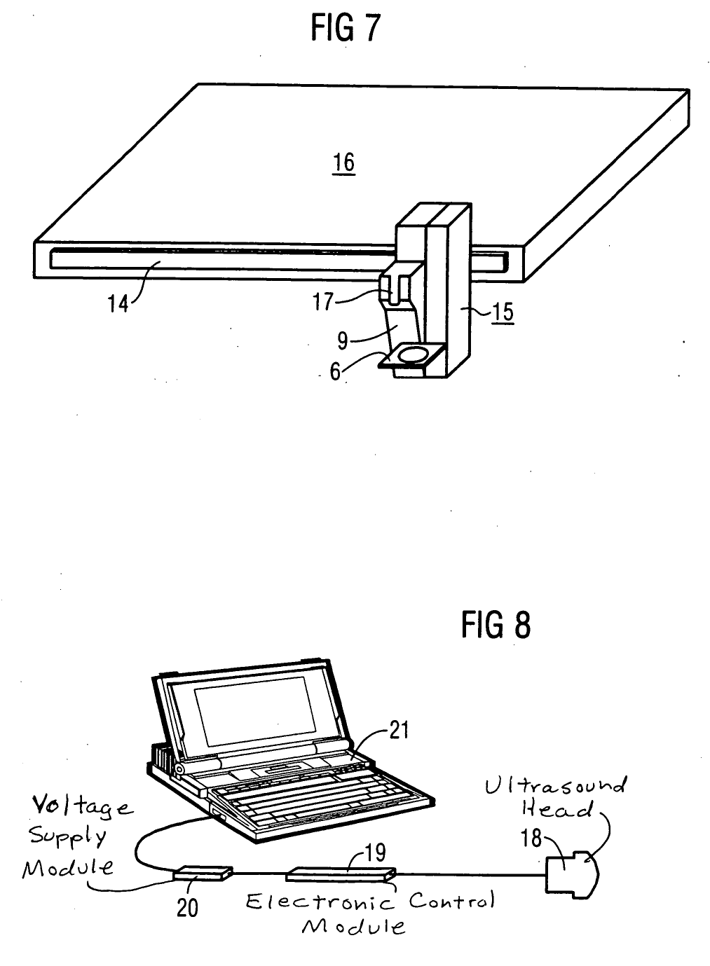

An ultrasound head mounting 15 with a base element 1 and an attachment element 3 is shown in FIG. 7 together with a patient positioning table 16, but the dimensions are not to scale. This mounting 15 can be moved to any arbitrary location of the table 2 where it is needed due to the attachment of the ultrasound head mounting 15 by means of clamps to the accessory rail 14 of the patient positioning table 16. The attachment module 2, as already noted, is equipped with an acceptor 9 for a small ultrasound head that exhibits the width of the chamber 8 for an electronics module covered by a cover 12. This acceptance 9 also has a slot 17 for easy insertion of the connection cable of the small ultrasound head.

FIG. 8 is an arrangement to control the large ultrasound head 18, which is connected via a cable with the aforementioned electronics module 19, which is connected via a voltage supply module 20 to a laptop 21 or notebook as an ultrasound PC. This voltage supply module 20, as already explained, is inserted into a chamber 4 of the base element 1, while the electronics module 19 is inserted into one of the chambers 8 of the first or second attachment element 2 or 3. The attachment elements 2 and 3 or the acceptor 9 or 11 for the ultrasound heads can be provided in the lower region with a hook (not shown) on which the excess cable of the ultrasound head can be hung to save space.

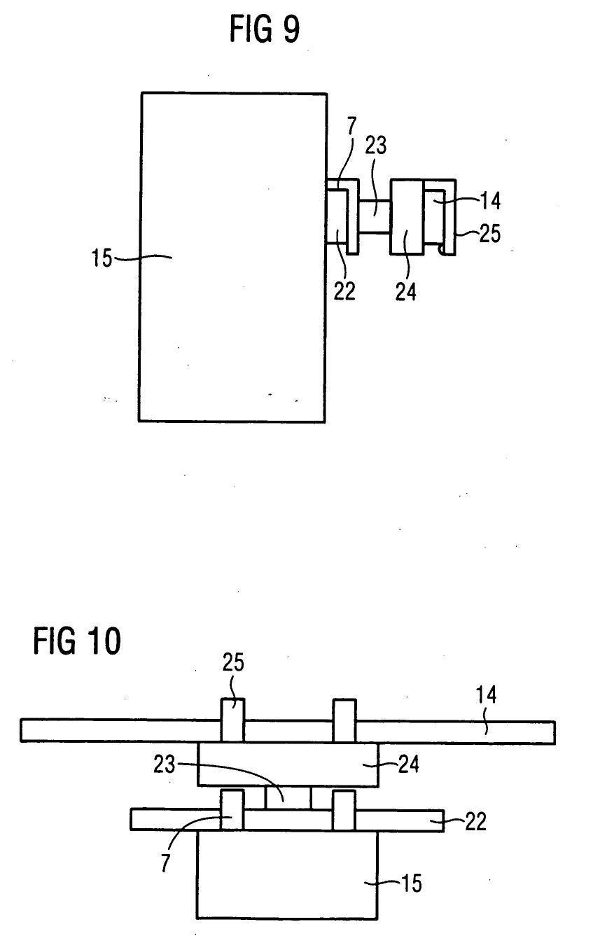

Since a patient positioning table, particularly for angiography, can execute tilting motions (which could lead to the ultrasound heads falling from their acceptor 9 or 11 given a rigidly attached ultrasound head mounting 15), a level-regulating adapter can be provided for the ultrasound head mounting 15. This is shown in FIG. 9. The ultrasound head mounting 15 engages, with the bracket 7, a mounting-side adapter rail 22 that is connected with a table-side adapter piece 24 via a rotation element 23 for level compensation. Like the ultrasound head mounting, this adapter piece 24 has a hook attachment 25 for the accessory rail 14. An ultrasound head mounting 15 also can be attached to a tilting table by means of this rotation adapter.

A plan view of the ultrasound head mounting with rotation adapter shown in FIG. 9 is shown in FIG. 10.

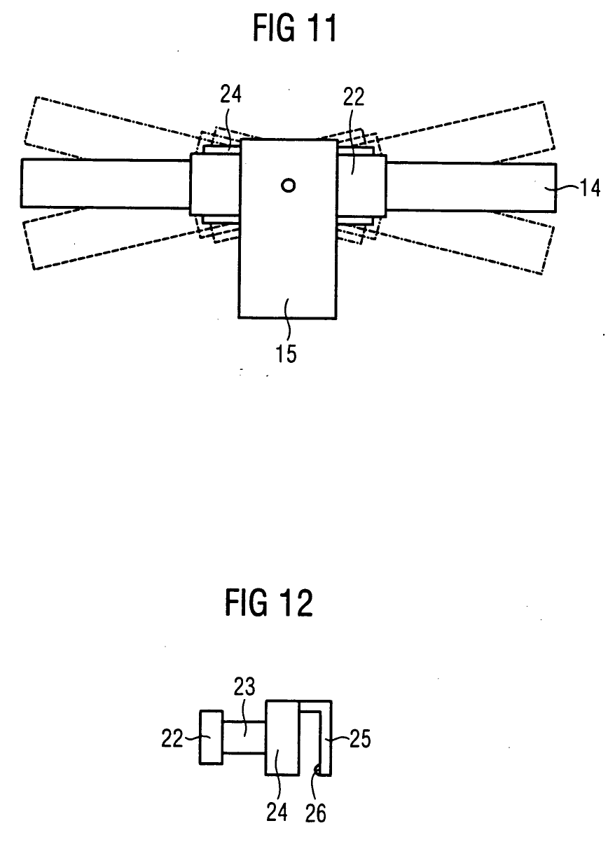

The functioning of the rotation adapter is shown in detail in FIG. 11. The accessory rail 14 is aligned horizontally and the ultrasound head mounting 15 is aligned vertically in the initial position of the patient table. If the patient positioning table is lowered to the left, the accessory rail 14 and the table-side adapter piece 23 are thus likewise rotated down and to the left in the position shown dashed. In contrast to this, if the table is lowered to the right, the position (shown dash-dot) for accessory rail 14 and table-side adapter piece 23 results. The ultrasound head mounting 15 always remains aligned vertically.

In FIG. 12, the rotation adapter with the mounting-side adapter rail 22, the rotation element 23, the table side adapter piece 24 and the hook attachment 25 for the accessory rail 14 are shown alone. The hook attachment 25 has a bolt 26 with spherical head to catch below the accessory rail 14. This allows the adapter to find secure purchase on the accessory rail 14.

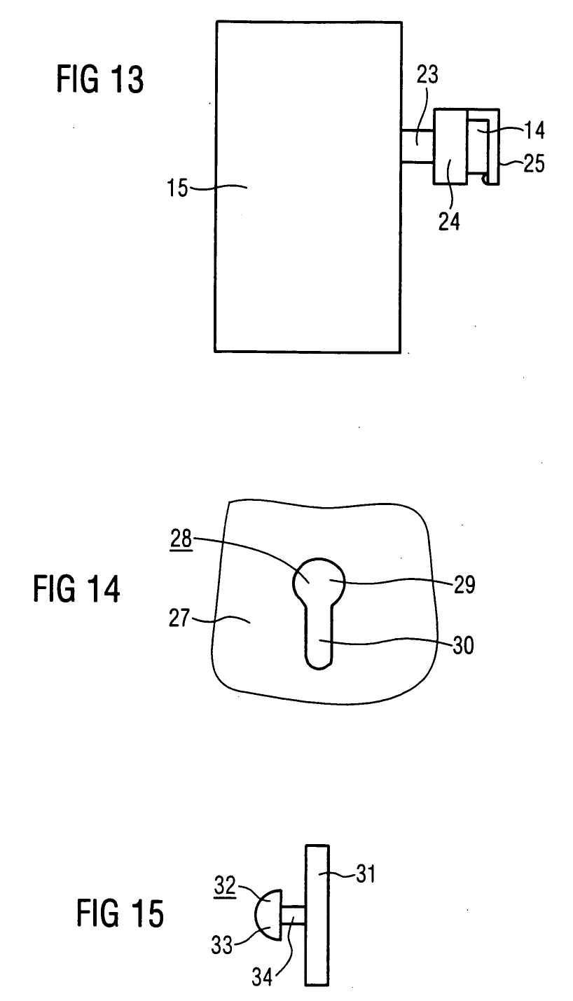

Alternatively, the ultrasound head mounting 15, as shown in FIG. 13, can be directly connected with the rotation element 23, such that the bracket 7 and the mounting-side adapter rails 22 are foregone since the ultrasound head mounting 15 is directly attached to the accessory rail 14 with the table-side adapter piece 24 and the hook attachment 25.

A further connection possibility of the individual elements 1 through 3, that can replace the previously specified swallowtail connection, is shown in FIGS. 14 and 15. FIG. 14 thus shows a side wall 27 of a first of the elements 1 through 3 in plan view that has a P-shaped cutout 28. This cutout is comprised of a bore 29 and a slot (oblong hole) 30 that exhibits a lesser width than the bore 29, behind which is a recess having the width of the bore diameter.

FIG. 15 is a side view of the counterpart that is arranged on the side wall 31 of a second of the elements 1 through 3 that is aligned to the side wall 27. This means that the side walls 27 and 31 lie opposite on an element. A knob 32 with a head 33 and a neck 34 is attached to the side wall 31. The head 33 exhibits a somewhat smaller diameter than the bore 29 so that it can be passed through this. The neck 34 exhibits a somewhat smaller diameter than the width of the of the slot 30 so that it can be inserted into this. For the purpose of the connection of the both elements, the knob 32 is inserted into the bore 29 until the head 33 completely penetrates the bore 29. The side walls 27 and 31 are then moved relative to one another such that the neck 34 of the knob 32 engages in the slot 30 and the elements 1 through 3 are held to one another by the head 33 abutting on the edges of the slot 30. Instead of the simple connection shown, the connection elements 28 and 32 can also be provided multiple times so that the stability increases. Since the actual purchase is produced by the clamps 7, a simple connection is possible.

A modern ultrasound apparatus has very small electronics boxes for the control of the ultrasound head and the acquisition of the image data. It is thus possible to adapt the ultrasound heads to the accessory rails of the patient table. This inventive ultrasound head mounting exhibits the following significant properties:
1. detachable elements that can be strung together in series for the assembly of one or more ultrasound heads on the table mounting,
2. a level-regulating adapter which allows tilting of the patient positioning table, and
3. a bracket for an ultrasound gel bottle.

The ultrasound head mounting is comprised of a base element 1 in which a voltage supply electronics module 20 is stored and one or more attachment elements 2 or 3 in which the respective ultrasound head electronic modules 19 and the ultrasound heads are stored. The base element 1 and the attachment elements 2 and 3 are modularly, laterally connected to one another via a swallowtail mechanism. Each element 1 through 3 possesses this swallowtail mechanism on both the left and the right sides and is always assembled on the same side to base element 1 or attachment element 2 or 3. This enables the connection of arbitrarily many attachment elements, allowing the combination of different ultrasound heads, for example for different medical applications. Given the application of an ultrasound head, the corresponding ultrasound head electronic module 19 must be connected to the voltage supply electronic module 20 with a cable. For sterility, this connection is located on the underside of the base element 1. The base element 1 has a second connection that enables the connection between the voltage supply electronic module and the ultrasound PC 21.

The electronic module 19 for each ultrasound head 18 is stored in a mounting that is attached to the accessory rail 14 of the patient positioning table 16. A ultrasound head mounting 9 or 11 the size of which is adapted to the size of the ultrasound head is built into the front of this mounting. A slot 13 for the passage of the ultrasound head cable is also provided under the ultrasound head mounting, and a bore is provided above to the back side of the mounting for the cable of the electronic module 19. A suspender is located outside of and underneath the ultrasound head electronic module mounting in order to be able to roll up the ultrasound head cable.

Instead of the swallowtail mechanism, a P-shaped cutout 28 on an element and a knob 32 on the neighboring element can be used.

There are angiography table options in which the patient positioning table 14 can be tilted up to 15° lengthwise and/or sideways. In order to compensate this tilting along the longitudinal axis so that it does not lead to the ultrasound heads falling off, an adapter 22 through 26 is provided. This adapter 22 through 26 is attached on the hook attachment of the ultrasound head mounting specified above according to FIG. 9, or is directly attached to the ultrasound head mounting according to FIG. 12, and is comprised of a rotation element 23 and a hook attachment 25. The ultrasound head mounting can be accommodated on the accessory rail 14 with the hook attachment 25, which is adapted to the size and shape of the accessory rail 14. The rotation element 23 serves for level compensation. When the patient positioning table 16 is tilted, this rotation element 23 rotates correspondingly, and thus the ultrasound head mounting as well, such that the ultrasound head mounting remains vertical and the ultrasound head cannot fall out.

The bracket 6 for an ultrasound gel bottle is attached to the base element 1. It is fashioned such that the bottle can be held at the neck of the bottle. This enables a better handling of the ultrasound gel bottle.

The application field for such mountings for ultrasound heads are interventional radiology, the operating room and the ultrasound department.

By means of the inventive apparatus, an ultrasound head can be operated directly at the patient table and be available to the physician at any time. This ultrasound head mounting 15 simplifies the implementation and improves the course of the interventional procedure. It can also be applied for an independent ultrasound apparatus with an ultrasound head on a gurney.

Instead of the shown swallowtail connection, swallowtail connections fashioned differently can be used. For example, the cross-section of the swallowtail connection can be V-shaped in a known manner.

Although modifications and changes may be suggested by those skilled in the art, it is the intention of the inventors to embody within the patent warranted hereon all changes and modifications as reasonably and properly come within the scope of their contribution to the art.

1. An apparatus for mounting at least one ultrasound head at a medical interventional facility, comprising:
a base element having a chamber adapted to receive a first electronic module, said base element having a mounting arrangement adapted to interact with equipment at a medical interventional facility for mounting the base element to the equipment; and
an attachment element removably engaged with said base element, said attachment element having a chamber adapted to accept a second electronic module, associated with an ultrasound head, and an acceptor adapted to receive and hold the ultrasound head.

2. An apparatus as claimed in claim 1 wherein said equipment is a patient positioning table having an accessory rail, and wherein said mounting arrangement is a bracket attachable to said accessory rail.

3. An apparatus as claimed in claim 1 wherein said base element and said attachment element have respective lateral sides, and wherein said base element and said attachment element are detachably connected to each other at said lateral sides.

4. An apparatus as claimed in claim 3 wherein said base element and said attachment element respectively carry complimentary components of a swallowtail mechanism at said lateral sides, said components of said swallowtail mechanism forming a detachable connection between said base element and said attachment element at said lateral sides.

5. An apparatus as claimed in claim 4 wherein one of said base element and said attachment element has a P-shaped cutout at the lateral side thereof, and wherein the other of said base element and said attachment element has a neck projecting from the lateral side thereof terminating with a knob, said knob fitting through a wider part of said P-shaped cutout and said neck fitting through a narrow part of said P-shaped cutout, forming a detachable connection between said base element and said attachment element at said lateral sides.

6. An apparatus as claimed in claim 1 wherein said chamber in said base element is adapted to receive a voltage supply module as said first electronic module.

7. An apparatus as claimed in claim 1 comprising a level-regulating adapter connected to one of said base element and said attachment element for maintaining said base element and said attachment element substantially horizontal when said equipment moves to a non-horizontal position.

8. An apparatus as claimed in claim 7 wherein said level-regulating adapter is detachably connected to said one of said base element and said attachment element.

9. An apparatus as claimed in claim 1 wherein said equipment is a patient positioning table having an accessory rail, and wherein said mounting arrangement comprises an adapter body having a hook adapted to fit over said accessory rail, said adapter body being connected to said base element via a rotational element allowing said base element to rotate relative to said adapter body for level-compensation.

10. An apparatus as claimed in claim 1 wherein one of said base element and said attachment element comprises a carrier adapted to receive and hold an ultrasound gel bottle.

11. An ultrasound apparatus for use in a medical interventional facility, comprising:
an ultrasound head;
an electronic control module for controlling said ultrasound head for acquiring image data;
a voltage supply for supplying operating voltage to said ultrasound head;
a computer for generating ultrasound images from said image data, said computer, said electronic control module, said voltage supply and said ultrasound head being connected in series via cable; and
an apparatus for mounting said ultrasound head to equipment at said medical interventional facility, said apparatus comprising a base element having a chamber containing said control module, said base element having a mounting arrangement adapted to interact with the equipment at a medical interventional facility for mounting the base element to the equipment, and an attachment element removably engaged with said base element, said attachment element having a containing said voltage supply, and an acceptor receiving and holding the ultrasound head.

12. An ultrasound apparatus as claimed in claim 1 1 wherein said equipment is a patient positioning table having an accessory rail, and wherein said mounting arrangement is a bracket attachable to said accessory rail.

13. An ultrasound apparatus as claimed in claim 11 wherein said base element and said attachment element have respective lateral sides, and wherein said base element and said attachment element are detachably connected to each other at said lateral sides.

14. An ultrasound apparatus as claimed in claim 13 wherein said base element and said attachment element respectively carry complimentary components of a swallowtail mechanism at said lateral sides, said components of said swallowtail mechanism forming a detachable connection between said base element and said attachment element at said lateral sides.

15. An ultrasound apparatus as claimed in claim 14 wherein one of said base element and said attachment element has a P-shaped cutout at the lateral side thereof, and wherein the other of said base element and said attachment element has a neck projecting from the lateral side thereof terminating with a knob, said knob fitting through a wider part of said P-shaped cutout and said neck fitting through a narrow part of said P-shaped cutout, forming a detachable connection between said base element and said attachment element at said lateral sides.

16. An ultrasound apparatus as claimed in claim 11 comprising a level-regulating adapter connected to one of said base element and said attachment element for maintaining said base element and said attachment element substantially horizontal when said equipment moves to a non-horizontal position.

17. An ultrasound apparatus as claimed in claim 16 wherein said level-regulating adapter is detachably connected to said one of said base element and said attachment element.

18. An ultrasound apparatus as claimed in claim 11 wherein said equipment is a patient positioning table having an accessory rail, and wherein said mounting arrangement comprises an adapter body having a hook adapted to fit over said accessory rail, said adapter body being connected to said base element via a rotational element allowing said base element to rotate relative to said adapter body for level-compensation.

19. An ultrasound apparatus as claimed in claim 11 wherein one of said base element and said attachment element comprises a carrier adapted to receive and hold an ultrasound gel bottle.
